(19) United States
(12) Patent Application Publication
(10) Pub. No.: US 2016/0376581 A1
(43) Pub. Date: Dec. 29, 2016

(54) METHOD FOR THE PURIFICATION OF TARGETED NUCLEIC ACIDS FROM BACKGROUND NUCLEIC ACIDS

(71) Applicant: IBIS BIOSCIENCES, INC., Carlsbad, CA (US)

(72) Inventors: Gregory Richmond, Carlsbad, CA (US); Jose R. Gutierrez, Carlsbad, CA (US); Steven A. Hofstadler, Cartsbad, CA (US)

(21) Appl. No.: 14/902,463

(22) PCT Filed: Jul. 2, 2014
(86) PCT No.: PCT/US14/45257

§ 371 Date: Dec. 31, 2015

Related U.S. Application Data
(63) Continuation of application No. 13/933,919, filed on Jul. 2, 2013.

(51) Int. Cl. C12N 15/10; C12Q 1/68; B01J 41/04

(52) U.S. Cl. CPC C12N 15/101; B01J 41/046; C12Q 1/689; C12Q 1/6895

(57) ABSTRACT

The present invention relates generally to the field of nucleic acid purification. In particular, provided herein are micro particles and micro particle clusters for selective anion exchange of nucleic acids, and methods and kits useful for this purpose.

### STATEMENT REGARDING FEDERALLY SPONSORED RESEARCH OR DEVELOPMENT

This invention was made with government support under 1 R01 CI000099-01 awarded by CDC. The government has certain rights in the invention.

### CROSS-REFERENCE TO RELATED APPLICATIONS

This application claims the benefit of U.S. application Ser. No. 13/933,919 filed Jul. 2, 2013, which is incorporated herein by reference in its entirety.

### FIELD OF THE INVENTION

The present invention relates generally to the field of nucleic acid purification. In particular, provided herein are micro particles and micro particle clusters for selective anion exchange of nucleic acids, and methods and kits useful for this purpose.

### BACKGROUND OF THE INVENTION

A variety of molecular biology, biochemical, and biophysical analysis techniques (e.g., mass spectrometry (e.g., electrospray ionization)) require relatively clean samples, for example, without contaminating non-target nucleic acids and/or without various contaminants (e.g., cationic salts, detergents, certain buffering agents, etc.).

Ethanol precipitation has been used to desalt PCR products for analysis as short oligonucleotides and salts are removed while the sample is concentrated (M. T. Krahmer, Y. A. Johnson, J. J. Walters, K. F. Fox, A. Fox and M. Nagpal, Electrospray Anal. Chem. 1999, 71, 2893-2900; T. Tsuneyoshi, K. Ishikawa, Y. Koga, Y. Naito, S. Baba, H. Terunuma, R. Arakawa and D. J. Prockop Rapid Commun. Mass Spectrom. 1997, 11, 719-722; and D. C. Muddiman, D. S. Wunschel, C. L. Liu, L. Pasatolic, K. F. Fox, A. Fox, G. A. Anderson and R. D. Smith Anal. Chem. 1996, 68, 3705-3712). In this method, the PCR product can be precipitated from concentrated ammonium acetate solutions, either overnight at 5° C. or over the course of 10-15 min with cold (−20° C.) ethanol. Unfortunately, a precipitation step alone is generally insufficient to obtain PCR products which are adequately desalted to obtain high-quality ESI spectra; consequently, precipitation is generally followed by a dialysis step to further desalt the sample (D. C. Muddiman, D. S. Wunschel, C. L. Liu, L. Pasatolic, K. F. Fox, A. Fox, G. A. Anderson and R. D. Smith Anal. Chem. 1996, 68, 3705-3712). While several researchers have successfully employed these methods to characterize a number of PCR products, the route to applying these methods in a robust and fully automated high-throughput manner is not obvious.

Commercial DNA purification kits may also be used in conjunction with traditional desalting techniques such as microdialysis (S. Hahner, A. Schneider, A. Ingendoh and J. Mosner Nucleic Acids Res. 2000, 28, e82/i-e82/viii; and A. P. Null, L. T. George and D. C. Muddiman J. Am. Soc. Mass Spectrom. 2002, 13, 338-344). Other purification techniques, such as gel electrophoresis followed by high-performance liquid chromatography or drop dialysis, or cation exchange using membranes or resins have also been used to obtain high-purity, desalted DNA for MS detection (L. M. Benson, S.-S. Juliane, P. D. Rodringues, T. Andy, L. J. Maher III and S. Naylor, In: The 47th ASMS Conference on Mass Spectrometry and Allied Topics, Dallas, Tex. (1999); C. G. Huber and M. R. Buchmeiser Anal. Chem. 1998, 70, 5288-5295; H. Oberacher, W. Parson, R. Muehlmann and C. G. Huber Anal. Chem. 2001, 73, 5109-5115; and C. J. Sciacchitano J. Liq. Chromatogr. Relat. Technol. 1996, 19, 2165-2178). Unfortunately, as with the techniques described above, the path toward a rapid and fully automated high-throughput implementation is not obvious.

Jiang and Hofstadler have developed and reported a single protocol for the purification and desalting of PCR products which employs commercially available pipette tips packed with anion exchange resin (Y. Jiang and S. A. Hofstadler Anal. Biochem. 2003, 316, 50-57). This protocol yields an ESI-MS-compatible sample and requires only 10:1 of crude PCR product. However, the method is cost-prohibitive when applied to high volume and high throughput processes such as the methods cited above for identification of unknown bioagents. Retail costs of using the commercially-obtained ZipTip™ AX (Millipore Corp. Bedford, Mass.) are estimated at $1.77 per plate well.

Anion exchange, wherein the anionic exchangers are selected from the group consisting of diethylaminoethyl (DEAE), quaternary methyl amine, and phosphate have been used to purify nucleic acid from cell lysate. However, discrimination between target and non-target DNA is not possible with existing known techniques.

Solution capture of nucleic acids such as those obtained from amplification reactions has enabled a rapid, cost-effective method of extracting and purifying these analytes for subsequent analysis by mass spectrometry. Since the nucleic acids and the anion exchange media are in solution, efficient capture of the nucleic acids is accomplished by vortexing, or other mixing methods. This has eliminated the need to pack the media in a column format which would require multiple passes of the nucleic acid solution over it to achieve high levels of recovery of nucleic acids. While longer columns require fewer passes, significant backpressure becomes a problem. The process of packing an anion exchange resin in a column or pipette tip format increases the cost associated with the procedure accordingly. Thus the use of solution capture for purification of PCR products for analysis by mass spectrometry has substantially reduced the cost associated with sample preparation by eliminating the need to pack, equilibrate, and test a column. The retail cost of the current procedure using a pipette tip packed with anion exchange resin exemplified by ZipTip™ AX (Millipore, Bedford, Mass.) is approximately $1.77 per pipette tip (for each sample). The estimated cost of solution capture of PCR products is $0.10 per sample and takes into account the combination of anion exchange resin and filter plate. Furthermore, the time required for solution capture purification of PCR products is approximately 10 minutes per 96 well plate in contrast to the previous method which employs the ZipTip™ AX pipette tips and requires approximately 20 minutes.

There remains a need for a method of purification of nucleic acids target size nucleic acids that is rapid, efficient and non-cost prohibitive.

### SUMMARY OF THE INVENTION

The present invention provides compositions and methods for selectively capturing and purifying targeted nucleic acids, for example, in a background containing large amounts of unwanted (e.g., non-target) nucleic acids. Capture and purification of nucleic acids is useful or necessary during, for example, processing of clinical and/or environmental specimens. The present invention relates to, inter alia, selective anion exchange of nucleic acids. In particular embodiments, microparticle clusters are provided for selective anion exchange of nucleic acids. Method of purifying nucleic acids with such microparticles, and kits comprising such microparticles are also provided. The nucleic acids are captured on microparticle clusters containing a weak anion exchange functional groups. A mechanism for removal of unwanted non-target nucleic acids from a matrix containing the microparticles is provided. In certain embodiments, after background nucleic acids are, for example, washed away, target nucleic acids are then selectively removed from the matrix.

In some embodiments, the manufacturing process for micro particle clusters creates irregularities (e.g., sub-micron sized pores or cavities) on the cluster surface and within the particle and/or clusters. The structural irregularities (e.g., pores) on the micro particles adhere desired target nucleic acid products (e.g., of a desired size or size range), due to size exclusion properties, while not adhering non-target nucleic acids (e.g., nucleic acids of non-target size (e.g., larger genomic nucleic acids)). In some embodiments, surface and/or internal irregularities (e.g., pores) are functionalized with a weak anion exchange functional group the bind nucleic acids.

In some embodiments, both target and non-target nucleic acids adhere to the porous microparticles, but conditions are provided in which target nucleic acids are selectively eluted from the weak anion surface while non-target (e.g., larger) nucleic acids are retained on the micro-particle. The binding and elution properties of the micro particle clusters are adjustable by controlling the conditions of an ambient medium.

In certain embodiments, compositions and methods provided herein allow a user to decrease large amounts of background nucleic acid from a sample (e.g., background nucleic acid generated during the processing of clinical and/or environmental specimens). In other embodiments, the invention allows selective capture of nucleic acids when large volumes of complex biological sample (e.g., blood) are processed to extract foreign nucleic acid (e.g. microorganism nucleic acid).

In some embodiments, the present invention provides compositions comprising a microparticle having a surface comprising cavities and/or other surface irregularities and/or an aggregate comprising two or more of said microparticles, which aggregate comprises an opening, wherein said surface, cavities, opening, and/or other surface irregularitiespores are: a) functionalized with a weak anion exchange functional group; and b) dimensioned for size exclusion of smaller nucleic acid molecules from larger nucleic acid molecules. In some embodiments, the larger nucleic molecules are >10 nucleotides, >15 nucleotides, >20 nucleotides, >30 nucleotides, >40 nucleotides, >50 nucleotides, >60 nucleotides, >70 nucleotides, >80 nucleotides, >90 nucleotides, >100 nucleotides, >150 nucleotides, >200 nucleotides, >300 nucleotides, >400 nucleotides, >500 nucleotides, >600 nucleotides, >700 nucleotides, >800 nucleotides, >900 nucleotides, >1000 nucleotides, etc. In some embodiments, larger nucleic acid molecules comprise or are derived from human genomic nucleic acid. In some embodiments, smaller nucleic acid molecules comprise or are derived from a microorganism nucleic acid. In some embodiments, compositions further comprise smaller nucleic acid molecules bound to the pores. In some embodiments, the microparticle is an iron particle. In some embodiments, the weak anion exchange functional group is an amine. In some embodiments, the amino is a primary, secondary, or tertiary alkyl amine. In some embodiments, the amine has a pKa of greater than 9. In some embodiments, the composition comprises a plurality of said microparticles. In some embodiments, the plurality of microparticles are in a resin. In some embodiments, the resin is on a solid surface. In some embodiments, the solid surface comprises a plate or column. In some embodiments, the plate or column comprises a wash buffer. In some embodiments, the wash buffer is configured to elute said smaller nucleic acid molecules from said microparticle, while leaving behind said larger nucleic acid molecules. In some embodiments, the wash buffer is compatible with mass spectrometry. In some embodiments, the wash buffer does not comprise a metal cation salt.

In some embodiments, a kit comprising a composition described herein is provided. In some embodiments, the kit comprises a wash buffer. In some embodiments, the wash buffer is configured to elute said smaller nucleic acid molecules from said microparticle, while leaving behind said larger nucleic acid molecules. In some embodiments, the wash buffer is compatible with mass spectrometry. In some embodiments, the wash buffer does not comprise a metal cation salt.

In some embodiments, a system comprising a composition described herein and an instrument for processing or analyzing a biological sample is provided. In some embodiments, the instrument comprises a nucleic acid amplification device. In some embodiments, the instrument comprises a nucleic acid sequencing device. In some embodiments, the instrument comprises a nucleic acid detection device. In some embodiments, the system comprises a control computer for automated processing of a plurality of said samples.

In some embodiments, the present invention provides methods of detecting a target nucleic acid in a sample comprising: a) exposing a sample to a microparticle having a surface comprising cavities and/or other surface irregularities and/or an aggregate comprising two or more of said microparticles, which aggregate comprises an opening, wherein said surface, cavities, opening, and/or other surface irregularities or pores are: i) functionalized with a weak anion exchange functional group; and ii) dimensioned for size exclusion of smaller nucleic acid molecules from larger nucleic acid molecules; b) binding smaller nucleic acid from said sample to said pores; c) isolating said smaller nucleic acid by selectively eluting from said pores; and d) detecting said smaller nucleic acid. In some embodiments, the sample is a blood, serum, or plasma sample.

In certain embodiments, the present invention is directed to solution capture methods of purifying a solution comprising one or more nucleic acids for subsequent analysis by electrospray mass spectrometry, or any other analysis, by adding an anion exchange resin to the solution and mixing to yield a suspension of the anion exchange resin in the solution wherein the nucleic acid binds to the anion exchange resin, isolating the anion exchange resin from the solution, washing the anion exchange resin to remove one or more contaminants with one or more wash buffers while retaining bound nucleic acid, eluting the nucleic acid, from the ion exchange resin with an elution buffer, and optionally, analyzing the nucleic acids by electrospray mass spectrometry.

The anion exchange resin may have a strong anion exchange functional group such as a quaternary amine or a weak anion exchange functional group such as, for example, polyethyleneimine, charged aromatic amine, diethylaminomethyl, or diethylaminoethyl. Such weak anion exchange resins comprise functional groups with pKₐ values of 9.0 or greater.

The present invention is further directed to kits for purification of nucleic acids comprising a filter plate comprising a plurality of wells or a tube rack comprising a plurality of tubes, an anion exchange resin, at least one anion exchange wash buffer and an ESI-MS-compatible elution buffer.

### BRIEF DESCRIPTION OF THE DRAWINGS

FIG. 1 demonstrates enrichment of target DNA in a high load background of non-target DNA using amine modified irregular shaped 1.5 μm magnetic beads.

FIG. 2 shows a graph demonstrating the yield of target amplicon eluted when a high DNA background is present is similar to the yield of amplicon eluted when no background DNA is present.

FIG. 3 shows graph depicting limits of detection for whole blood spiked with (A) K. pneunomoniae (KPC) and (B) E. faecium (VRE) performed by Plex-ID using amine enrichment beads.

FIG. 4 shows a graph depicting a limit of detection comparison performed for four bacterial organisms between samples with 3 and 12 μg of background human DNA.

FIG. 5 is a process diagram outlining the steps of the present invention beginning with the addition and mixing of anion exchange resin into the sample of nucleic acids (100). The resin is then isolated from the solution (110) and washed with an appropriate wash buffer to remove contaminants from the resin (120) after which, the nucleic acids are eluted from the resin by an electrospray ionization (ESI)-compatible elution buffer, which makes possible the final step of analysis of the nucleic acids by ESI-mass spectrometry (140).

FIG. 6 is a comparison of ESI-MS spectra for purified PCR products obtained by purification with ZipTips™ (top panel) and by the solution capture purification method of the present invention. The comparison indicates that purification by the solution capture method is equally effective as the previously validated method which employs ZipTips™.

FIG. 7 is an ESI-FTICR mass spectrum of an amplification product obtained by a PCR reaction on a section of the genome of Staphylococcus aureus which was purified via the use of amine terminated supraparamagnetic beads as described in Example 10.

### DESCRIPTION OF EMBODIMENTS

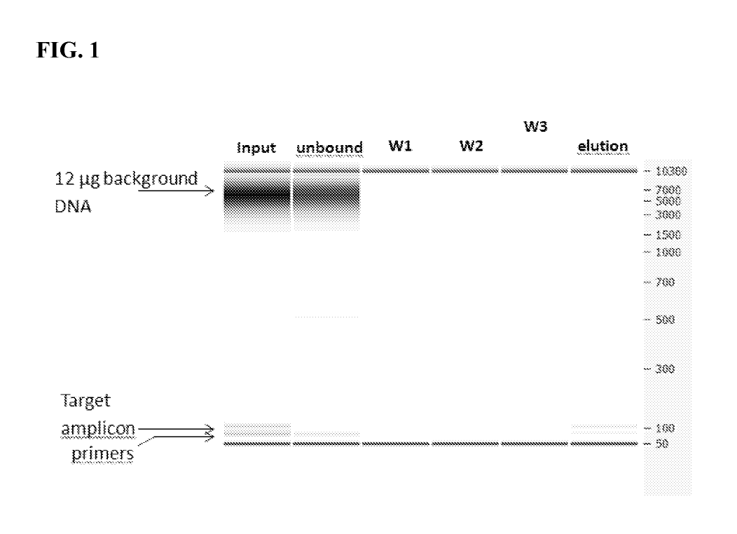

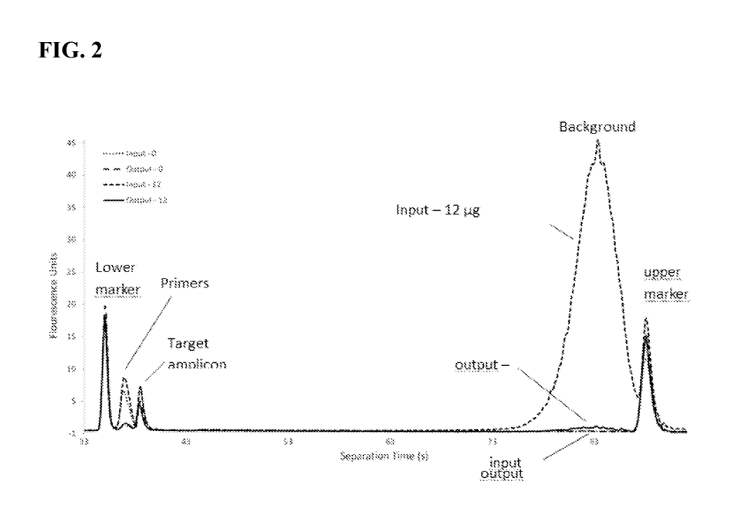

In certain embodiments, compositions and methods are provided that included a weak anion surface on a micro particle or micro particle clusters for selective anion exchange of nucleic acid. Removal of unwanted non-target genomic NA molecules (See FIGS. 1 and 2) involves 1) preferentially adhering desired short target nucleic acid products to the micro particle clusters, and pores within the cluster, and keeping behind at least a portion, but preferably the majority, of unwanted larger genomic nucleic acid material; and 2) preferentially eluting the desired target nucleic acid products from the weak anion surface on micro particle material while retaining on the micro-particle as much unwanted larger genomic nucleic acid material as possible. The binding and elution properties of the micro particle clusters with respect to nucleic acids are adjustable by controlling the conditions of an ambient medium (e.g., buffer, salt, pH, etc.).

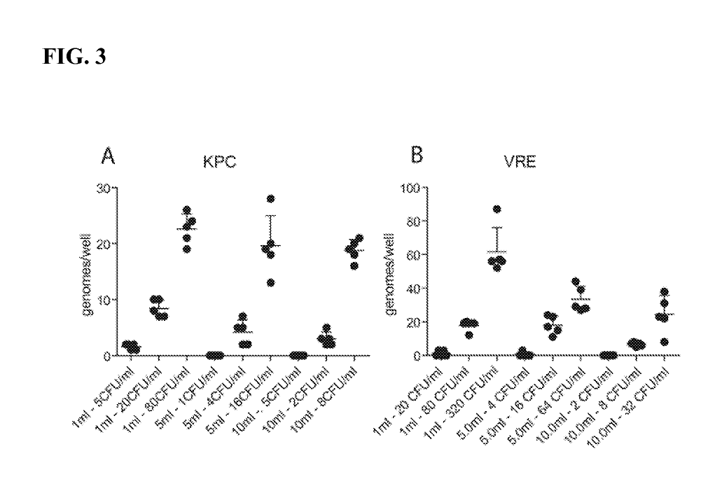

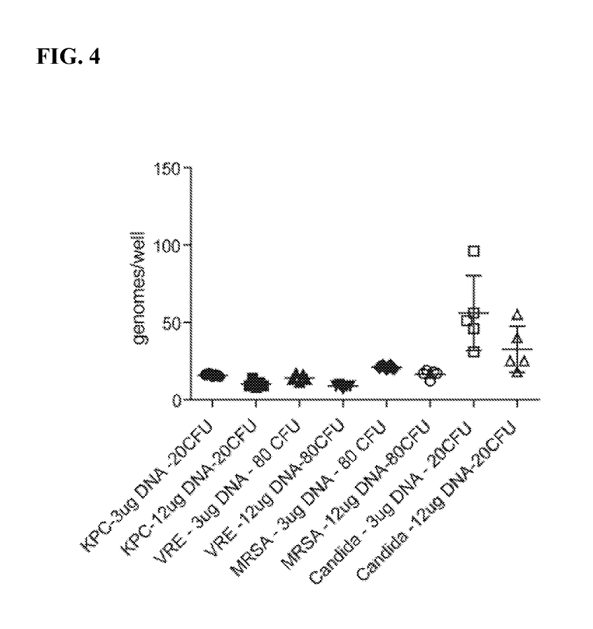

One useful aspect of this invention is the ability to decrease large amounts of background nucleic acid that is generated during the processing of clinical and/or environmental specimens. In various embodiments, target nucleic acids is purified when, for example, large volumes of blood are processed (e.g., to extract foreign nucleic acid (e.g. microorganism DNA). Excess amounts of human genomic DNA are unnecessarily extracted during the extraction of relatively small amounts foreign DNA. Frequently, for some analysis platforms (e.g. in mass spectrometry), at least a portion of the background human DNA must be removed to allow target DNA analysis. The present invention has been shown to selectively capture target nucleic acid products, with an enrichment factor of, for example, 75 fold, from PCR reactions that originate from clinical specimens. As a result of applying this approach, it has been discovered that an equivalent number of colony forming units (CFU) of a microorganism can be detected in blood specimens regardless of whether the specimen consisted of 1 ml, 5 ml, or 10 ml of blood, which contain increasing amounts of background DNA (See FIGS. 3 and 4). In some embodiments, the enriched sample is amenable to mass spectrometry analysis, which is often incompatible to many sample types.

In some embodiments, the technology is suitable for purification of nucleic acid amplification products (e.g. PCR products) from primers, primer-dimers, and non-polynucleotide components of the reaction, and selectively separates, or enriches, for a particular polynucleotide component (e.g. target product nucleic acid) from other polynucleotide components found in amplification reactions (e.g. template and background genomic nucleic acid, primers). Unlike existing nucleic acid purification methods, the technology provides inexpensive and efficient purification of target nucleic acids, and is amenable to automation and high throughput.

In some embodiments, the technology utilizes a combination of size exclusion (e.g., as a result of surface and/or interior irregularities (e.g., pores and/or cavities)) and anion exchange (e.g., as a result of functionalized surface and/or interior) to selectively bind, release, and purify target nucleic acids (e.g., nucleic acids of a selected size range); although the present invention is not limited to any particular mechanism of action and an understanding of the mechanism of action is not necessary to practice the present invention. In some embodiments, target nucleic acids are under a given size threshold. For example, in some embodiments, target nucleic acids are <20 nucleotides, <30 nucleotides, <40 nucleotides, <50 nucleotides, <60 nucleotides, <70 nucleotides, <80 nucleotides, <90 nucleotides, <100 nucleotides, <150 nucleotides, <200 nucleotides, <300 nucleotides, <400 nucleotides, <500 nucleotides, <600 nucleotides, <700 nucleotides, <800 nucleotides, etc. In some embodiments, target nucleic acids are over a given size threshold. For example, in some embodiments, target nucleic acids are >5 nucleotides, >10 nucleotides, >15 nucleotides, >20 nucleotides, >25 nucleotides, >30 nucleotides, >40 nucleotides, >50 nucleotides, >60 nucleotides, >70 nucleotides, >80 nucleotides, >90 nucleotides, >100 nucleotides, etc. In some embodiments, target nucleic acids are a range of sizes with both upper and lower thresholds.

In some embodiments, microparticles are magnetic, contain functional groups that allow for anion exchange of nucleic acids, and comprise irregular surface features (e.g., pores) that allow for size-selective adherence and/or release of nucleic acids. In some embodiments, magnetic particles allow, for example, manipulation of microparticles (e.g., with or without adhered nucleic acid).

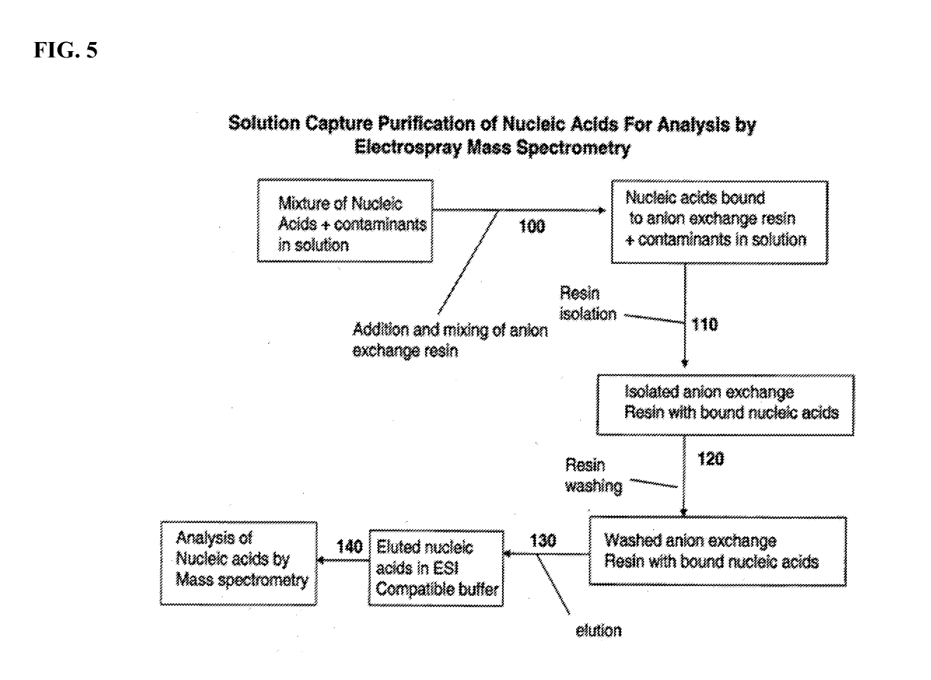

One embodiment of the method of solution capture purification of nucleic acids for analysis by mass spectrometry, for example, is outlined in FIG. 5. The methods described herein can be used for other types of analysis, in addition to mass spectrometry as known to those skilled in the art. The methods comprise the following steps: Addition and mixing of an anion exchange resin into a solution of nucleic acids (100), isolating the anion exchange resin from the solution (110), washing the anion exchange resin to remove contaminants (120), eluting the nucleic acids, (free of contaminants) from the anion exchange resin (130), and, optionally, analyzing the nucleic acid by ESI mass spectrometry.

In some embodiments, a strong cation exchange functional group, such as a quaternary amine for example, is employed as the functional group of the anion exchange resin. Additional strong anion exchange functional groups are known to those skilled in the art.

In other embodiments, a weak anion exchange functional group is a suitable anion exchange functional group, such as polyethyleneimine, charged aromatic amine, diethylaminomethyl, or diethylaminoethyl, for example, are employed as the functional group of the anion exchange resin. Such functional groups have pKₐ values of 9.0 or greater. Commercial products of weak anion exchange resin include, but are not limited to; Baker PEI, Baker DEAM, Dionex ProPac™ WAX, Millipore PEI, Applied Biosystems Poros™ PI.

In some embodiments, the mixing of the anion exchange resin into the solution of nucleic acids is effected by repeated pipetting, vortexing, sonication, shaking, or any other method that results in suspension of the anion exchange resin in the solution containing the nucleic acids.

In some embodiments, dry anion exchange resin is added directly to the solution of nucleic acids or contained within a microtube or the well of a micro filter plate into which the solution of nucleic acids is added prior to mixing. In other embodiments, the anion exchange resin is pre-hydrated and added directly to the solution of nucleic acids or contained within a microtube or a well of a microfilter plate into which the solution of nucleic acids is added prior to mixing.

In some embodiments, the anion exchange resin which contains bound nucleic acids is isolated from the solution by filtration. Filtration can be effected, for example, using a filter plate in a 96- or 384-well format which enables high-throughput purification of multiple samples, or in any other container or plurality of containers equipped with a filter. Other well format plates can also be used. Membranes useful for filtration include but are not limited to those composed of the following materials: polytetrafluoroethylene (PTFE), polyvinyldifluoro (PVDF), polypropylene, polyethylene, glass fiber, polycarbonate and polysulfone. Filtering may be accomplished by vacuum, centrifugation, or positive pressure displacement with fluids or gases, or any other method that effects the isolation of the anion exchange resin from the solution. Methods of filtering are well known to those skilled in the art.

In some embodiments, the anion exchange resin comprises an anion exchange functional group which is linked to magnetic beads. Such an arrangement enables a simpler isolation step (110) by eliminating the need for centrifugation, vacuum or positive pressure displacement which would necessitate the removal of the plate or microtube tube from the liquid handler deck. Instead, a magnetic field can be activated to compress the magnetic bead resin so that liquid can be aspirated off by the liquid handler. Methods of using magnetic beads to effect isolation of biomolecules are well known to those skilled in the art.

In some embodiments, the anion exchange resin which contains bound nucleic acids is washed to remove one or more contaminants. Contaminants include, but are not limited to: proteins such as reverse transcriptase and restriction enzymes, polymers, salts, buffer additives, or any of the various components of an amplification reaction such as polymerases nucleotide triphosphates or any combination thereof. Depending on the composition of the contaminants in the nucleic acid solution, more than one wash buffer may be useful for removal of contaminants. Washing of the anion exchange resin can be effected with aqueous solutions of ammonium acetate in the millimolar range from about 20 mM to about 500 mM NH₄OAc or with about 20 mM to about 500 mM NH₄HCO₃. Washing with about 10% to about 50% methanol, about 20% to about 50% methanol, or about 10% to about 30% methanol is useful as a final wash step. Methanol can be replaced by other suitable alcohols known to those skilled in the art.

In some embodiments, elution of nucleic acids from the anion exchange resin is accomplished using an ESI-compatible solution at alkaline pH of about pH 9 or greater such as an aqueous solution of about 2% to about 8% ammonium hydroxide or an aqueous solution of about 10 mM to about 50 mM, or 25 mM piperidine, about 10 mM to about 50 mM, or 25 mM imidazole and about 30% methanol or other suitable alcohol. As defined herein, an ESI-compatible solution is a solution which does not have a detrimental effect on the function of an electrospray (ESI) source.

As used herein, the term “about” means+10% of the term being modified. Thus, for example, “about” 10 mM means 9 to 11 mM.

In another embodiment, the present invention also provides kits for purification of nucleic acids by the solution capture method of the present invention. In some embodiments, the kit may comprise a sufficient quantity of anion exchange resin. In some embodiments, the anion exchange resin is a weak anion exchange resin such as one of the following commercially available weak anion exchange resins: Baker polyethyleneimine resin, Baker diethylaminomethyl resin, Dionex ProPac™ WAX, Millipore polyethyleneimine, and Applied Biosystems POROS™ PI.

In some embodiments, the kit may comprise a filter plate such as a 96- or 384-well filter plate or a microtube rack comprising a plurality of micro filter tubes.

In some embodiments, dry anion exchange resin is pre-loaded into the wells of a filter plate or microtube rack and can be either pre-hydrated or in the dry (powder) form.

The kit may also comprise a filter plate comprising a plurality of wells or a tube rack comprising a plurality of tubes, an anion exchange resin, at least one anion exchange wash buffer and an ESI-MS-compatible elution buffer.

In one embodiment, the kit may comprise a 96 or 384 well plate containing either pre-hydrated anion exchange resin or dry anion exchange resin, a second 96 or 384 well sample mixing plate, a 96 or 384 well filter plate, a resin treatment buffer, one or more wash buffers, and an ESI-compatible elution buffer.

In one embodiment, the nucleic acid solution is a PCR product prepared for identification of an unknown bioagent and contained in an individual well of a 96 well sample plate on the deck of an automated liquid handler. The liquid handler is the cornerstone for many laboratory processes associated with drug discovery and high throughput screening. The dispensing and aspiration functions of liquid handlers are used to perform solvent/reagent additions, dilutions, plate replications consolidation, redistribution and other microplate-based tasks and typically use disposable pipette tips for transferring liquids. Programming of liquid handlers to perform the various liquid handling tasks of this embodiment is well within the capabilities of one with ordinary skill in the art without undue experimentation.

The liquid handler is programmed to transfer and mix a predetermined volume of a suspension of anion exchange resin into the well containing the PCR product. The resin suspension can be contained in a resin source container such a 96 well plate and transferred to the PCR product plate by the liquid handler. Mixing is performed by the liquid handler via repeated dispensation and aspiration of the PCR-resin mixture and binding of nucleic acids to the resin occurs at this stage. Next, the liquid handler transfers the PCR product-resin mixture from the 96 well plate to a 96 or 384 well filter plate. At this stage, the filter plate can be removed from the liquid handler deck and the resin can be isolated from the solution by centrifugation or positive pressure displacement before returning the filter plate to the liquid handler deck.

The resin containing bound nucleic acids is then washed one or more times with an appropriate wash solution such as about 100 mM NH₄HCO₃ with the liquid handler pipetting the wash solution into the filter plate, followed by centrifugation, vacuum, or positive pressure displacement followed by one or more washes with about 20% to about 50% methanol before returning the filter plate containing the resin and bound nucleic acids to the liquid handler deck.

Finally, the nucleic acids are eluted from the resin with an ESI compatible elution buffer such as an aqueous solution of about 25 mM piperidine, about 25 mM imidazole and about 50% methanol. This ESI compatible buffer may also optionally contain an internal standard used to calibrate the ESI mass spectrometer during the subsequent ESI mass spectrometry analysis.

In order that the invention disclosed herein may be more efficiently understood, examples are provided below. It should be understood that these examples are for illustrative purposes only and are not to be construed as limiting the invention in any manner. Throughout these examples, molecular cloning reactions and other standard recombinant DNA techniques were carried out according to methods described in Maniatis et al., Molecular Cloning—A Laboratory Manual, 2^{nd} ed. Cold Spring Harbor Press (1989), using commercially available reagents except where otherwise noted.

### EXAMPLES

### Example 1

### Enrichment of Target DNA in a High Load Background of Non-Target DNA Using Amine Modified Irregular Shaped 1.5 Cpm Magnetic Beads

Capillary electrophoresis analysis (See FIG. 1) was used to measure the polynucleotide components of a post-PCR reaction that contained 12 μg of human DNA before enrichment (Input). A four minute incubation in the presence of amine magnetic beads allowed preferential binding of target amplicon, while a majority of the background DNA and primers did not bind (unbound). After three washes (W1,W2,W3), the target amplicons were eluted from the beads while the remaining background DNA did not elute in appreciable quantities relative to its starting amount (elution).

### Example 2

### Target Elution in High DNA Background

The yield of target amplicon eluted in a high DNA background was compared to elution when no background DNA was present (See FIG. 2). Capillary electrophoresis analysis was used to measure the polynucleotide components of a post-PCR reaction that contained 0 or 12 μg of human DNA before enrichment (Input—0, Input—12, respectively). After primary amine anion exchange, target amplicon yields are similar for both conditions, but the target amplicon has been enriched since the amount of background DNA (Output—12) has decreased substantially relative to input levels.

### Example 3

### Limit of Detection, K. pneunomoniae (KPC) and E. faecium (VRE)

Limit of detection were analyzed for whole blood spiked with K. pneunomoniae (KPC) and E. faecium (VRE) performed by Plex-ID using amine enrichment beads (See FIG. 3). Donor blood samples (1 ml, 5 ml and 10 ml) were spiked with different levels of K. pneunomoniae and E. faecium colony forming units (CFUs): 5 CFU, 20 CFU, 80 CFU and 20 CFU, 80 CFU and 320 CFU respectively. Samples were lysed by bead beating, total DNA was extracted by KingFisher technology, followed by PCR amplification and Plex-ID testing. Bang beads were used for DNA clean up before samples spray on the TOF mass spectrometer. The bacterial genomes were identified for each sample (genomes/well) for five repetitions/sample group. The estimated limit of detection (more than 95% positive detections/sample group) for K. pneunomoniae across the different tested volumes is a total 20 CFU (20 CFU/ml for 1 ml; 4 CFU/ml for 5 ml and 2 CFU/ml for 10 ml). The estimated limit of detection for E. faecium is 80 CFU (80 CFU/ml for 1 ml, 16 CFU/ml for 5 ml and 8 CFU/ml for 10 ml). Total background DNA/PCR reaction was measured by Nanodrop; for the tested blood samples the background DNA/PCR reaction were 0.7 μg for 1 ml, 3 μg for 5 ml and 6 μg for 10 ml.

### Example 4

### Limit of Detection Comparison

Limit of detection comparison performed for four bacterial organisms between samples with 3 and 12 μg of background human DNA. 20 CFU K. pneunomoniae (KPC), 80 CFU E. faecium (VRE), 80 CFU S. aureus (MRSA) and 20 CFU C. albicans (Candida) were spiked in whole blood, lysed by bead beating and extracted by KingFisher technology followed by PCR amplification and Plex-ID testing (FIG. 4). Bangs beads were used by Plex-ID systems to enrich target DNA before they got sprayed on the TOF mass spectrometer. Total DNA, quantified by Nanodrop, gave a background human DNA/PCR reaction of 3 μg and 12 μg. All the tested spikes show 100% detection for the two compared background DNA levels.

### Example 5

### Nucleic Acid Isolation and PCR

In one embodiment, nucleic acid is isolated from the organisms and amplified by PCR using standard methods prior to BCS determination by mass spectrometry. Nucleic acid is isolated, for example, by detergent lysis of bacterial cells, centrifugation and ethanol precipitation. Nucleic acid isolation methods are described in, for example, Current Protocols in Molecular Biology (Ausubel et al.) and Molecular Cloning; A Laboratory Manual (Sambrook et al.). The nucleic acid is then amplified using standard methodology, such as PCR, with primers which bind to conserved regions of the nucleic acid which contain an intervening variable sequence as described below.

General Genomic DNA Sample Prep Protocol:

Raw samples are filtered using Supor-200 0.2 μM membrane syringe filters (VWR International). Samples are transferred to 1.5 ml eppendorf tubes pre-filled with 0.45 g of 0.7 mm Zirconia beads followed by the addition of 350 μl of ATL buffer (Qiagen, Valencia, Calif.). The samples are subjected to bead beating for 10 minutes at a frequency of 19 l/s in a Retsch Vibration Mill (Retsch). After centrifugation, samples are transferred to an S-block plate (Qiagen) and DNA isolation is completed with a BioRobot 8000 nucleic acid isolation robot (Qiagen).

Swab Sample Protocol:

Allegiance S/P brand culture swabs and collection/transport system are used to collect samples. After drying, swabs are placed in 17×100 mm culture tubes (VWR International) and the genomic nucleic acid isolation is carried out automatically with a Qiagen Mdx robot and the Qiagen QIAamp DNA Blood BioRobot Mdx genomic preparation kit (Qiagen, Valencia, Calif.).

### Example 6

### Mass Spectrometry

The mass spectrometer used is a Bruker Daltonics (Billerica, Mass.) Apex II 70e electrospray ionization Fourier transform ion cyclotron resonance mass spectrometer (ESI-FTICR-MS) that employs an actively shielded 7 Tesla superconducting magnet. All aspects of pulse sequence control and data acquisition were performed on a 1.1 GHz Pentium II data station running Bruker's Xmass software. 20 μL sample aliquots were extracted directly from 96-well microtiter plates using a CTC HTS PAL autosampler (LEAP Technologies, Carrboro, N.C.) triggered by the data station. Samples were injected directly into the ESI source at a flow rate of 75 μL/hr. Ions were formed via electrospray ionization in a modified Analytica (Branford, Conn.) source employing an off axis, grounded electrospray probe positioned ca. 1.5 cm from the metalized terminus of a glass desolvation capillary. The atmospheric pressure end of the glass capillary is biased at 6000 V relative to the ESI needle during data acquisition. A counter-current flow of dry N₂/O₂ was employed to assist in the desolvation process. Ions were accumulated in an external ion reservoir comprised of an rf-only hexapole, a skimmer cone, and an auxiliary gate electrode, prior to injection into the trapped ion cell where they were mass analyzed.

Spectral acquisition was performed in the continuous duty cycle mode whereby ions were accumulated in the hexapole ion reservoir simultaneously with ion detection in the trapped ion cell. Following a 1.2 ms transfer event, in which ions were transferred to the trapped ion cell, the ions were subjected to a 1.6 ms chirp excitation corresponding to 8000-500 m/z. Data was acquired over an m/z range of 500-5000 (1M data points over a 225K Hz bandwidth). Each spectrum was the result of co-adding 32 transients. Transients were zero-filled once prior to the magnitude mode Fourier transform and post calibration using the internal mass standard. The ICR-2LS software package (G. A. Anderson, J. E. Bruce (Pacific Northwest National Laboratory, Richland, W A, 1995) was used to deconvolute the mass spectra and calculate the mass of the monoisotopic species using an “averaging” fitting routine (M. W. Senko, S. C. Beu, F. W. McLafferty, J. Am. Soc. Mass Spectrom. 1995, 6, 229) modified for DNA. Using this approach, monoisotopic molecular weights were calculated.

### Example 7

### Procedure for Semi-Automated Purification of a PCR Mixture Using Commercially Available ZipTips™

For pre-treatment of ZipTips™ AX (Millipore Corp. Bedford, Mass.), the following steps were programmed to be performed by an Evolution™ P3 liquid handler (Perkin Elmer) with fluids being drawn from stock solutions in individual wells of a 96-well plate (Marshall Bioscience): loading of a rack of ZipTips™ AX; washing of ZipTips™ AX with 15 μl of 10% NH₄OH/50% methanol; washing of ZipTips™ AX with 15 μl of water 8 times; washing of ZipTips™ AX with 15 μl of 100 mM NH₄OAc.

For purification of a PCR mixture, 20 μl of crude PCR product was transferred to individual wells of a MJ Research plate using a BioHit™ multichannel pipette. Individual wells of a 96-well plate were filled with 300 μl of 40 mM NH₄HCO₃. Individual wells of a 96-well plate were filled with 300 μl of 20% methanol. An MJ research plate was filled with 10 μl of 4% NH₄OH. Two reservoirs were filled with deionized water. All plates and reservoirs were placed on the deck of the Evolution™ P3 (EP3) pipetting station in pre-arranged order. The following steps were programmed to be performed by an Evolution™ P3 pipetting station: aspiration of 20 μl of air into the EP3 P50 head; loading of a pre-treated rack of ZipTips™ AX into the EP3 P50 head; dispensation of the 20 μl NH₄HCO₃ from the ZipTips™ AX; loading of the PCR product into the ZipTips™ AX by aspiration/dispensation of the PCR solution 18 times; washing of the ZipTips™ AX containing bound nucleic acids with 15 μl of 40 mM NH₄HCO₃ 8 times; washing of the ZipTips™ AX containing bound nucleic acids with 15 μl of 20% methanol 24 times; elution of the purified nucleic acids from the ZipTips™ AX by aspiration/dispensation with 15 μl of 4% NH₄OH 18 times. For final preparation for analysis by ESI-MS, each sample was diluted 1:1 by volume with 70% methanol containing 50 mM piperidine and 50 mM imidazole.

### Example 8

### Procedure for Semi-Automated Purification of a PCR Mixture with Solution Capture

For pre-treatment of ProPac™ WAX weak anion exchange resin, the following steps were performed in bulk: sequential washing three times (10:1 volume ratio of buffer to resin) with each of the following solutions: (1) 1.0 M formic acid/50% methanol (2) 20% methanol (3) 10% NH₄OH (4) 20% methanol (5) 40 mM NH₄HCO₃ (6) 100 mM NH₄OAc. The resin is stored in 20 mM NH₄OAc/50% methanol at 4° C.

Corning 384-well glass fiber filter plates were pre-treated with two rinses of 250 μl NH₄OH and two rinses of 100 μl NH₄HCO₃.

For binding of the PCR product nucleic acids to the resin, the following steps were programmed to be performed by the Evolution™ P3 liquid handler: addition of 0.05 to 10 μl of pre-treated ProPac™ WAX weak anion exchange resin (30 μl of a 1:60 dilution) to a 50 μl PCR reaction mixture (80 μl total volume) in a 96-well plate; mixing of the solution by aspiration/dispensation for 2.5 minutes; and transfer of the solution to a pre-treated Corning 384-well glass fiber filter plate. This step was followed by centrifugation to remove liquid from the resin and is performed manually, or under the control of a robotic arm.

The resin containing nucleic acids was then washed by rinsing three times with 200 μl of 100 mM NH₄OAc, 200 μl of 40 mM NH₄HCO₃ with removal of buffer by centrifugation for about 15 seconds followed by rinsing three times with 20% methanol for about 15 seconds. The final rinse was followed by an extended centrifugation step (1-2 minutes).

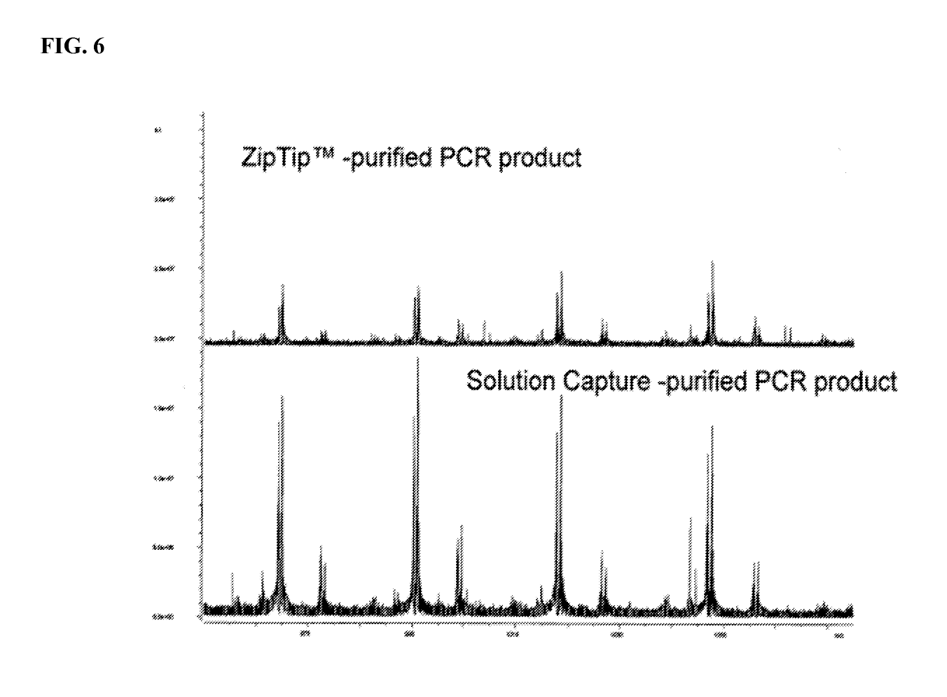

Elution of the nucleic acids from the resin was accomplished by addition of 40 μl elution/electrospray buffer (25 mM piperidine/25 mM imidazole/35% methanol and 50 nM of an internal standard oligonucleotide for calibration of mass spectrometry signals) followed by elution from the 384-well filter plate into a 384-well catch plate by centrifugation. The eluted nucleic acids in this condition were amenable to analysis by ESI-MS (See FIG. 6). The time required for purification of samples in a single 96-well plate using a liquid handler is approximately five minutes.

### Example 9

### Comparison of the ZipTips™ Purification Method with the Solution Capture Method

To investigate the efficacy of the solution capture method of the present invention, the ESI-MS analysis results obtained for PCR products purified with the solution capture method (Example 8) were compared with the ZipTips™ method outlined in Example 7.

Bacillus anthracis DNA was isolated and amplified by PCR using a primer pair that amplifies a section of the lef gene of B. anthracis ranging from residues 756-872. Shown in FIG. 6 is a comparison of ESI-MS spectra for purified PCR products obtained by purification with ZipTips™ (top panel) and by the solution capture purification method of the present invention. The comparison indicates that purification by the solution capture method is equally effective as the previously validated method which employs ZipTips™. However, purification by solution capture represents a significant cost savings and is more efficient. As stated by Jiang and Hofstadler, purification of PCR products using ZipTips™ “yields an ESI-compatible sample (96-well plate) in less than 20 minutes.” The solution capture method of the present invention yields an ESI-compatible sample in approximately five minutes. The retail cost of the current procedure using a pipette tip packed with anion exchange resin exemplified by ZipTip™ AX (Millipore, Bedford, Mass.) is approximately $1.77 per pipette tip (for each sample). The estimated cost of solution capture of PCR products is $0.10 per sample and takes into account the combination of anion exchange resin and filter plate.

### Example 10

### Solution Capture of Nucleic Acids with Ion Exchange Resin-Magnetic Beads

To confirm the efficacy of carrying out solution capture of nucleic acids with ion exchange resin linked to magnetic beads, 25 μl of a 2.5 mg/mL suspension of BioClon amine terminated supraparamagnetic beads were added to 25 to 50 μl of a PCR reaction containing approximately 10 pM of a typical PCR amplicon such as an amplicon obtained from broad priming of Staphylococcus aureus. The above suspension was mixed for approximately 5 minutes by vortexing or pipetting, after which the liquid was removed after using a magnetic separator to separate out the beads. The beads containing bound PCR amplicon were then washed 3× with 50 mM ammonium bicarbonate/50% MeOH or 100 mM ammonium bicarbonate/50% MeOH, followed by three more washes with 50% MeOH. The bound PCR amplicon was eluted with 25 mM piperidine, 25 mM imidazole, 35% MeOH, plus peptide calibration standards.

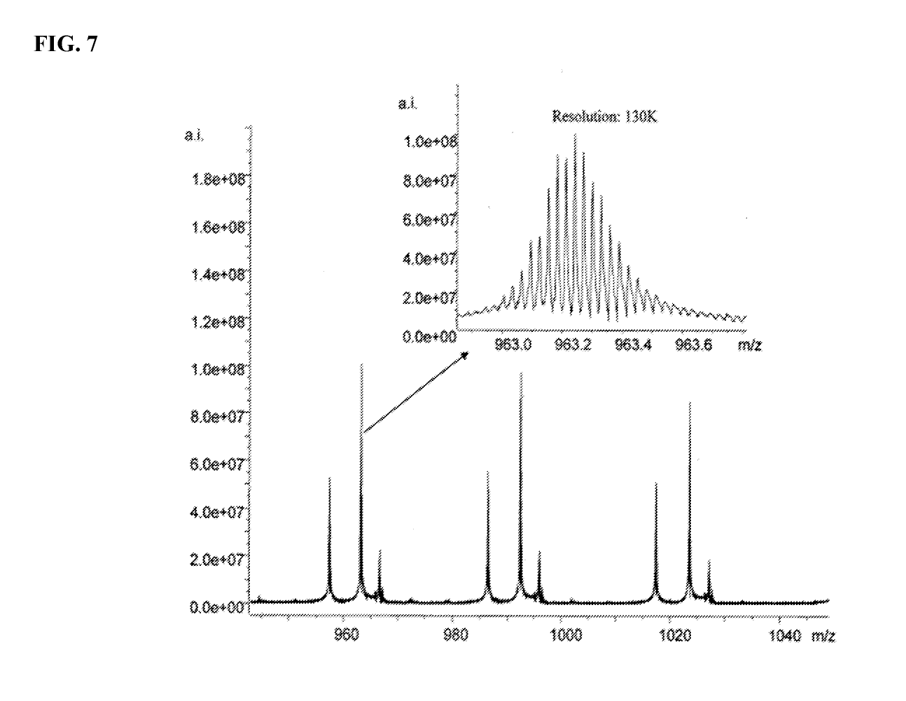

The eluate was then analyzed by ESI-FTICR electrospray ionization mass spectrometry (ESI). The ESI-FTICR mass spectrum is shown in FIG. 7 and indicates that effective purification of the Staphylococcus aureus amplicon has been achieved through the use of magnetic beads as a separation tool.

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

1. A composition comprising a microparticle having a surface comprising cavities and/or other surface irregularities and/or an aggregate comprising two or more of said microparticles, which aggregate comprises an opening, wherein said surface, cavities, opening, and/or other surface irregularities or pores are: a) functionalized with a weak anion exchange functional group; and b) dimensioned for size exclusion of smaller nucleic acid molecules from larger nucleic acid molecules.

2. The composition of claim 1, wherein said larger nucleic molecules are greater than 200 nucleotides in length.

3. The composition of claim 1, wherein said larger nucleic acid molecules comprise or are derived from human genomic nucleic acid.

4. The composition of claim 1, wherein said smaller nucleic acid molecule comprise or are derived from a microorganism nucleic acid.

5. The composition of claim 1, further comprising smaller nucleic acid molecules bound to said pores.

6. The composition of claim 1, wherein said microparticle is an iron particle.

7. The composition of claim 1, wherein said weak anion exchange functional group is an amine.

8. The composition of claim 7, wherein said amino is a primary, secondary, or tertiary alkyl amine.

9. The composition of claim 7, wherein said amine has a pKa of greater than 9.

10. The composition of claim 1, comprising a plurality of said microparticles.

11. The composition of claim 10, wherein said plurality of microparticles are in a resin.

12. The composition of claim 11, wherein said resin is on a solid surface.

13. The composition of claim 12, wherein said solid surface comprises a plate or column.

14. The composition of claim 13, wherein said plate or column comprises a wash buffer.

15. The composition of claim 14, wherein said wash buffer is configured to elute said smaller nucleic acid molecules from said microparticle, while leaving behind said larger nucleic acid molecules.

16. The composition of claim 14, wherein said wash buffer is compatible with mass spectrometry.

17. The composition of claim 16, wherein said wash buffer does not comprise a metal cation salt.

18-27. (canceled)

28. A method of detecting a target nucleic acid in a sample comprising: a) exposing a sample to a composition of claim 1; b) binding smaller nucleic acid from said sample to said pores; c) isolating said smaller nucleic acid by selectively eluting from said pores; and d) detecting said smaller nucleic acid.

29. The method of claim 28, wherein said sample is a blood, serum, or plasma sample.

30. The method of claim 28, wherein said target nucleic acid is from K. pneunomoniae, E. faecium, S. aureus, or C. albicans.
